(19) United States
(12) Patent Application Publication
(10) Pub. No.: US 2021/0081570 A1
(43) Pub. Date: Mar. 18, 2021

(54) SECURING PHYSICAL OBSERVATIONS AND ENABLING PROOF OF PHYSICAL WORK

(71) Applicant: Hypergiant Industries, Inc., Austin, TX (US)

(72) Inventors: Benjamin Edward Lamm, Dallas, TX (US); Andrew Thomas Busey, Austin, TX (US); Christopher Dillinger, Austin, TX (US); Daniel David Haab, Austin, TX (US); Davis Michael Saltzgiver, Austin, TX (US)

(21) Appl. No.: 17/021,826

(22) Filed: Sep. 15, 2020

Related U.S. Application Data
(60) Provisional application No. 62/901,428, filed on Sep. 17, 2019.

(51) Int. Cl. G06F 21/64; H04L 9/32; H04L 9/30; C12M 1/00

(52) U.S. Cl. CPC G06F 21/64; H04L 9/3271; C12M 21/02; H04L 9/30; H04L 9/3297; H04L 9/3247

(57) ABSTRACT

Systems and methods for verifying physical measurements taken for bioreactors are disclosed. Physical measurements may be taken using one or more sensors coupled to a bioreactor. The sensors may measure properties of the bioreactor or properties of materials within the bioreactor. Verifying the physical measurements may be implemented to enable proof of physical work being completed by the bioreactor.

### PRIORITY CLAIM

The present application claims priority to U.S. Prov. Appl. No. 62/901,428, filed Sep. 17, 2019, which is incorporated by reference herein in its entirety.

### BACKGROUND

### Technical Field

The present disclosure relates generally to devices and techniques for securely generating observations of physical properties. More particularly, embodiments disclosed herein relate to devices, such as photobioreactors, configured to securely generate measurements of physical properties that enable the generation of proof of physical work, such as effort or progress made using the device.

### Description of Related Art

Generally speaking, computationally verifying that physical observations have been reliably made and are acceptably trustworthy is a difficult challenge. For example, a user may claim to have done some useful physical work by producing some quantity of a substance or object having defined physical characteristics. One might readily verify the user's claim if one had direct physical access to the user's work environment and output in order to perform any of a variety of tests. However, direct human inspection does not readily scale.

As an alternative to direct inspection, the user's work environment and/or output may be assessed remotely, possibly automatically, via sensors. By performing measurements and computationally evaluating those measurements, verification of users' claims of physical work may be scaled enormously.

Remote verification of physical work presents problems of trust and reliability, however. Users may attempt to corrupt the process by manipulating the measurement environment to produce expected results, or by modifying sensor measurements before they are transmitted for evaluation. If these issues are not addressed, efforts to scale verification of physical work through computational techniques may fail.

### BRIEF DESCRIPTION OF THE DRAWINGS

Features and advantages of the methods and apparatus of the embodiments described in this disclosure will be more fully appreciated by reference to the following detailed description of presently preferred but nonetheless illustrative embodiments in accordance with the embodiments described in this disclosure when taken in conjunction with the accompanying drawings in which:

FIG. 1 depicts an isometric view of an embodiment of a bioreactor.

FIG. 2 depicts a block diagram of an embodiment of a bioreactor.

FIG. 3 depicts a diagram of an embodiment of a sensor.

FIG. 4 depicts a flow diagram of an embodiment of a method of operation of a bioreactor using a sensor.

FIG. 5 depicts a flow diagram of an embodiment of a method of operation of a bioreactor.

While embodiments described in this disclosure may be susceptible to various modifications and alternative forms, specific embodiments thereof are shown by way of example in the drawings and will herein be described in detail. It should be understood, however, that the drawings and detailed description thereto are not intended to limit the embodiments to the particular form disclosed, but on the contrary, the intention is to cover all modifications, equivalents and alternatives falling within the spirit and scope of the appended claims. The headings used herein are for organizational purposes only and are not meant to be used to limit the scope of the description. As used throughout this application, the word “may” is used in a permissive sense (i.e., meaning having the potential to), rather than the mandatory sense (i.e., meaning must). Similarly, the words “include”, “including”, and “includes” mean including, but not limited to.

Various units, circuits, or other components may be described as “configured to” perform a task or tasks. In such contexts, “configured to” is a broad recitation of structure generally meaning “having circuitry that” performs the task or tasks during operation. As such, the unit/circuit/component can be configured to perform the task even when the unit/circuit/component is not currently on. In general, the circuitry that forms the structure corresponding to “configured to” may include hardware circuits and/or memory storing program instructions executable to implement the operation. The memory can include volatile memory such as static or dynamic random access memory and/or nonvolatile memory such as optical or magnetic disk storage, flash memory, programmable read-only memories, etc. The hardware circuits may include any combination of combinatorial logic circuitry, clocked storage devices such as flops, registers, latches, etc., finite state machines, memory such as static random access memory or embedded dynamic random access memory, custom designed circuitry, programmable logic arrays, etc. Similarly, various units/circuits/components may be described as performing a task or tasks, for convenience in the description. Such descriptions should be interpreted as including the phrase “configured to.” Reciting a unit/circuit/component that is configured to perform one or more tasks is expressly intended not to invoke 35 U.S.C. § 112(f) interpretation for that unit/circuit/component.

The scope of the present disclosure includes any feature or combination of features disclosed herein (either explicitly or implicitly), or any generalization thereof, whether or not it mitigates any or all of the problems addressed herein. Accordingly, new claims may be formulated during prosecution of this application (or an application claiming priority thereto) to any such combination of features. In particular, with reference to the appended claims, features from dependent claims may be combined with those of the independent claims and features from respective independent claims may be combined in any appropriate manner and not merely in the specific combinations enumerated in the appended claims.

This specification includes references to “one embodiment” or “an embodiment.” The appearances of the phrases “in one embodiment” or “in an embodiment” do not necessarily refer to the same embodiment, although embodiments that include any combination of the features are generally contemplated, unless expressly disclaimed herein. Particular features, structures, or characteristics may be combined in any suitable manner consistent with this disclosure.

### DETAILED DESCRIPTION OF EMBODIMENTS

Photobioreactors are reactors that utilize a light source to support the growth of phototrophic microorganisms in a controlled, artificial environment. Photobioreactors may be used to support photosynthetic growth of various different organisms using carbon dioxide and light. Examples of organisms that may been grown using photobioreactors include algae (e.g., macroalgae and/or microalgae), plants, mosses, cyanobacteria, and purple bacteria.

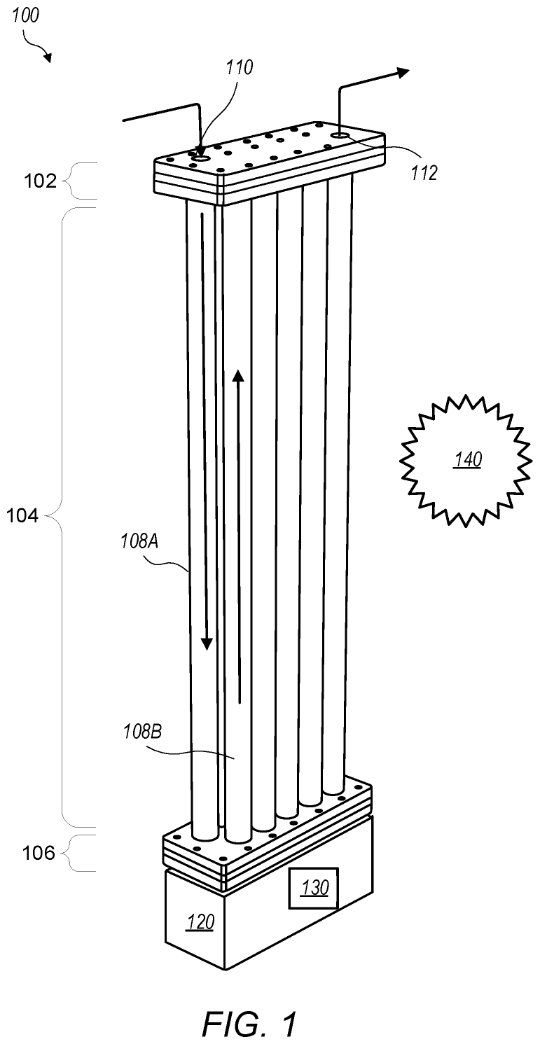

FIG. 1 depicts a perspective view of an embodiment of bioreactor 100. In certain embodiments, bioreactor 100 is a modular bioreactor. For example, bioreactor 100 may be coupled to one or more additional bioreactors to form up a larger bioreactor. In such embodiments, bioreactor 100 may include connections that allow multiple bioreactors to be coupled together. In some embodiments, multiple bioreactors 100 may be coupled together in series to form a single, larger bioreactor. In some embodiments, multiple bioreactors 100 may be coupled together in parallel to provide multiple parallel outputs of organisms.

In certain embodiments, bioreactor 100 includes top manifold 102, tube section 104, and bottom manifold 106. Tube section 104 may include a plurality of tubes 108 coupled between top manifold 102 and bottom manifold 106. Tubes 108 may have walls made of glass, plastic, or any other material that is substantially transparent to a desired spectrum of light (e.g., visible spectrum light). Top manifold 102 and bottom manifold 106 may direct (e.g., route) the flow of fluid through tubes 108 (e.g., direct fluid flow from one tube to the next).

In the illustrated embodiment, top manifold 102 and bottom manifold 106 direct fluid in a single direction through each of tubes 108 between inlet 110 and outlet 112. Thus, as shown by the arrows in FIG. 1, fluid may enter bioreactor 100 at inlet 110, go down first tube 108A, then up second tube 108B, and continue this pattern to outlet 112. Directing fluid through each of tubes 108 may route the fluid in a linear way and make one continuous flow path for fluid through the tubes (e.g., fluid flows in series through the tubes). Providing the one continuous flow path through tubes 108 in bioreactor 100 may maximize the surface area in contact with the fluid in the bioreactor for the growth of biological organisms in the bioreactor.

In some embodiments, routing fluid through inlet 110, tubes 108, and outlet 112 as shown in FIG. 1 may provide modularity for the design of bioreactor 100 and allow the bioreactor to be coupled to one or more additional bioreactors as part of a group of bioreactors. In certain embodiments, both inlet 110 and outlet 112 are positioned in a single manifold (e.g., top manifold 102). For example, with an even number of tubes 108, inlet 110, and outlet 112 may be positioned in the same manifold. Other embodiments with odd numbers of tubes may also be contemplated. In such embodiments with odd numbers of tubes 108, inlet 110 and outlet 112 may be positioned in different manifolds (e.g., the inlet is in top manifold 102 and the outlet is in bottom manifold 106).

Bioreactor 100 may be used to grow different types of biological organisms. In certain embodiments, bioreactor 100 is used to grow algae. The algae may be macroalgae and/or microalgae. Other biological organisms that may be grown using bioreactor 100 include, but are not limited to, plants, mosses, and bacteria (e.g., cyanobacteria or purple bacteria). Top manifold 102 and bottom manifold 106 provide structures that hold tubes 108 as close together as possible to produce a small footprint for bioreactor 100.

In certain embodiments, as shown in FIG. 1, utility system 120 is positioned near or coupled to a manifold in bioreactor 100. In certain embodiments, utility system 120 is attached to or positioned in a structure (e.g., a housing or cabinet) used to support the manifolds and tubes to provide a modular system for the bioreactor. Utility system 120 may include devices and/or apparatus that are used to facilitate growth of biological organisms in bioreactor 100. Examples of devices and/or apparatus included in utility system include, but are not limited to, fluid circulators (e.g., pumps), reservoirs (e.g., tanks), sensors, gas sources, nutrient (feedstock or raw material) feeders, and cleaning devices.

In certain embodiments, a reservoir in utility system 120 is in fluid communication with tubes 108 (e.g., through inlet 110 on a manifold (e.g., top manifold 102)). The reservoir may be a source of fluid and feedstock used for the growth of biological organisms in tubes 108. In some embodiments, a fluid circulator (e.g., a pump) is coupled to or placed in the reservoir. The fluid circulator may move fluid and feedstock to tubes 108 from the reservoir. In some embodiments, the reservoir may be an open reservoir that allows carbon dioxide to be pulled from the surrounding air. One or more sensors 130, described herein, may also be placed in the reservoir.

In certain embodiments, a harvester is included in utility system 120. The harvester may, for example, be coupled to outlet 112 on a manifold (e.g., top manifold 102) and be in fluid communication with tubes 108 through the outlet. The harvester may be used to harvest biomass (e.g., a mass of biological organisms) grown in tubes 108. In certain embodiments, utility system 120 is coupled to inlet 110 and outlet 112 on a manifold (e.g., top manifold 102). Tubes and/or valves may be used to couple utility system 120 to the manifold. In some embodiments, pumps or other fluid circulators in utility system provide pressure to create mixed flow in tubes 108 (e.g., mixing of biomass and fluid in the tubes). Mixing in tubes 108 may be used to inhibit settling of biomass in the manifolds and/or to promote growth of biomass in the tubes.

In certain embodiments, bioreactor 100 includes light source 140. Light source 140 may be any light source capable of providing light in wavelengths suitable for growth of a desired biological organism in bioreactor 100. For example, light source 140 may be fluorescent lights or LED lights capable of UV or near-UV radiation. In some embodiments, light source 140 is attached or included as part of a structure (e.g., a housing or cabinet) used to support the manifolds and tubes of bioreactor 100. In some embodiments, light source 140 is external to the structure used to support the manifolds and tubes of bioreactor 100.

A bioreactor, as described herein, represents a scalable, distributed model for performing useful or physical work. As used herein, “physical work” refers to any change in physical state of some object or system that (a) is produced through the application of resources (e.g., time, energy, consumable inputs) and (b) is capable of measurement. One particular context in which the problem of securing physical observations and enabling proof of physical work may arise is that of evaluating the state of a bioreactor. Users may choose to deploy and maintain bioreactors and may be compensated in some fashion based on their productivity. For example, users may be compensated based on the quantity and/or quality of bioreactor output. In order to remotely assess users' work, the bioreactor may include a variety of sensors (e.g., sensors 150) that attempt to measure various parameters that collectively characterize that work. But users might seek to falsify such measurements in order to receive compensation for work that was not performed. For example, a sensor might attempt to verify that the color of the growth medium within the bioreactor was consistent with the presence of successfully grown algae. A user might attempt to falsify this measurement by adding colorant to the growth medium, producing the expected result without the corresponding physical work. Alternatively, the user might attempt to alter the data captured by the sensor before it is transmitted.

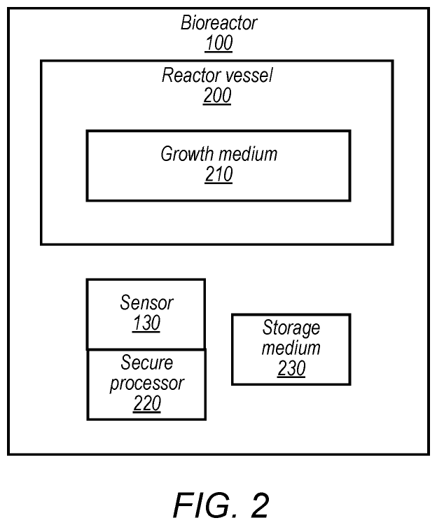

FIG. 2 depicts a block diagram of an embodiment of bioreactor 100. Bioreactor 100 includes elements that facilitate secure physical observations and the establishment of trust, so that proof of physical work can be reliably evaluated. In certain embodiments, as shown in FIG. 2, bioreactor 100 includes reactor vessel 200 that includes growth medium 210. Reactor vessel 200 may include, for example, tubes 108 (shown in FIG. 1). Bioreactor 100 additionally includes sensor 130 that is tightly coupled to secure processor 220. Any number of sensors 150 may be provided, although for simplicity, only one is shown. In some embodiments, each instance of sensor 130 may have a respective instance of secure processor 220, whereas in other embodiments, access to multiple sensors 130 may be performed through a single instance of secure processor 220. Bioreactor 100 may further include storage medium 230. These various elements will be discussed in greater detail below.

It is noted that bioreactor 100 may include numerous other components in various embodiments. For example, depending on the design, bioreactor 100 may also include pumps, light sources, heating and/or cooling elements, various ports or automated devices for the insertion of biological source material (e.g., algae), nutrients, or the like, and/or other elements. For simplicity of illustration, these elements are omitted.

During operation, growth medium 210 is placed into reactor vessel 200 and inoculated with the appropriate biological material, such as algae, that is to be produced. The various controllable features of bioreactor 100 are then initialized to produce conditions suitable for growth. These conditions may be maintained by a controller that may be implemented either within secure processor 220 or elsewhere within bioreactor 100.

In certain embodiments, one or more sensors 130 are included in bioreactor 100. Sensors 130, generally speaking, may measure any suitable operating property or physical property within reactor vessel 200 or within bioreactor 100. In certain embodiments, sensors 130 are placed in or coupled to a secondary reservoir coupled to tubes 108. For example, sensors 130 may be located in utility system 120, as shown in FIG. 1. In some embodiments, sensors 130 are provided into tubes 108 using access ports in the manifolds.

Operating properties assessed by sensors 130 may include, but not be limited to, amount of biomass, flow rate, temperature, pressure, pH, and photon detection. In various embodiments, sensors 130 may measure properties such as resistivity, salinity, temperature, and/or density within growth medium 210. Sensors 130 may measure one or more optical properties within growth medium 210, such as color and/or transmissivity/transparency. Sensors 130 may perform various types of chemical analyses, for example, to identify certain types and quantities of dissolved gases (e.g., carbon dioxide, oxygen, nitrogen) or to identify types and quantities of particular chemical compounds such as nutrients and/or waste products. To perform chemical analyses, sensors 130 may, for example, perform spectrographic analysis. Sensors 130 may also perform more sophisticated chemical analyses, such as DNA analysis. In various embodiments, some instances of sensors 130 may perform multiple different types of physical measurements. As mentioned above, numerous instances of different types of sensors 130 may be employed, and redundant sensors 130 may be used to improve robustness.

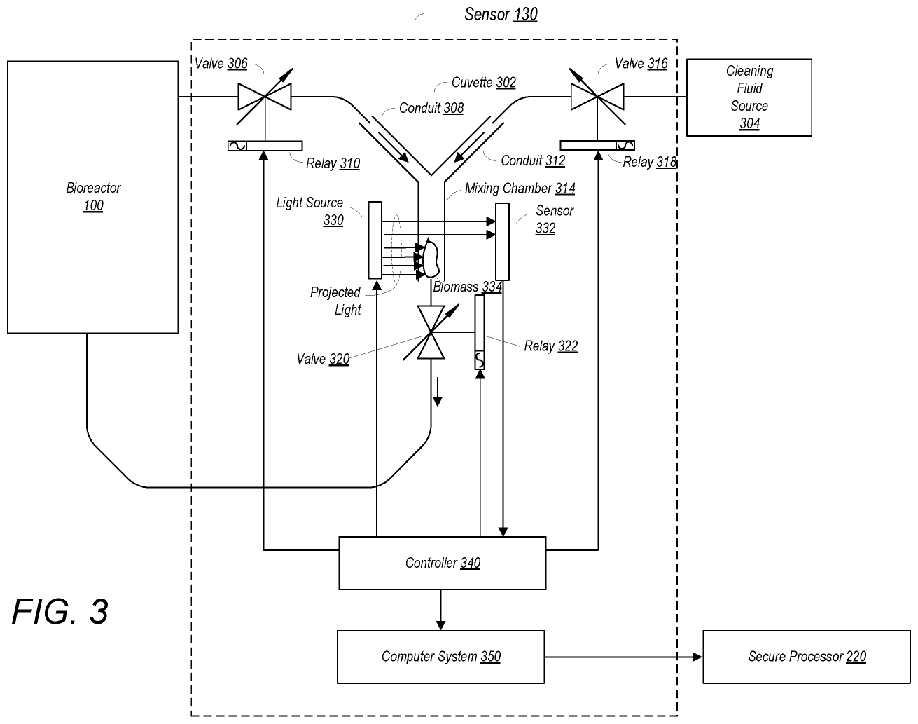

FIG. 3 depicts a diagram of an embodiment of sensor 130. Sensor 130 is one type of sensor that may be implemented to assess properties in bioreactor 100. In the illustrated embodiment, sensor 130 includes cuvette 302, which is arranged to receive fluid from bioreactor 100, as well as receiving fluid from cleaning fluid source 304. First valve 306 may be coupled between bioreactor 100 and conduit 308 of cuvette 302. Valve 306, in this embodiment, is controllable via relay 310, which can be used to open and close the valve based on signals received thereby. When open, valve 306 allows fluid containing a portion of the biomass to flow from bioreactor 100 into conduit 308.

Cuvette 302 of the illustrated embodiment includes both conduit 308 and conduit 312, which are commonly coupled to mixing chamber 314. As noted above, conduit 308 is arranged to convey fluid from bioreactor 100 into the mixing chamber 314. Conduit 312 may be arranged to convey cleaning fluid into mixing chamber 314. Similar to the arrangement discussed above, valve 316 is coupled between cleaning fluid source 304 and cuvette 302. Valve 316 may be opened and closed by relay 318 in response to a signal received by the latter.

Various types of cleaning fluid may be used for cleaning operations. In one embodiment, water is used as the cleaning fluid, although other types of fluids (which may include water) can be used as desired. Furthermore, in some embodiments, cleaning fluid source 304 may be arranged to apply pressure to the cleaning fluid when conveyed into cuvette 302. This may be useful, for example, when the types of biomass received by cuvette 302 have a tendency to build up on its inner surfaces.

Another valve 320 and relay 322 combination in the embodiment shown is arranged to cause draining of cuvette 302. Relay 322 may be used to open valve 320 when it is desirable to drain fluid from cuvette 302. When it is desired that cuvette 302 retain fluid, valve 320 is closed. After a sample of biomass is taken, valve 320 may be opened to drain cuvette 302 while returning the fluid containing the biomass back to bioreactor 100. During cleaning operations, mixing chamber 314 may be flushed with cleaning fluid while valve 320 coupled thereto may remain open. Although not shown here, additional valves and pipes may be present to allow draining of the cleaning fluid to another location not associated with bioreactor 100.

Sampling of the biomass may be performed using light source 330 and light sensor 332 arranged near mixing chamber 314 of cuvette 302. Light source 330 may be any suitable type of light source, such as one or more light emitting diodes (LEDs), lasers (which can be coherent for specific wavelengths), or any other suitable type of illuminator. In various embodiments, the light source 330 projects light at wavelengths that are absorbable by the biomass. In one embodiment, the biomass comprises one or more strains of algae that absorb light in the visible spectrum range of 380-750 nanometers (nm). Accordingly, light source 330 used in such an embodiment is selected to project light primarily within the visible spectrum. More generally, the wavelengths projected by light source 330 in various embodiments may be selected based on the type of biomass to be processed in bioreactor 100, and may include all or part of the visible spectrum.

Additional embodiments having different types of light sources are possible and contemplated. For example, an RGB (Red-Green-Blue) LED using pulse width modulation (PWM) is contemplated as a light source for another embodiment. The wavelengths used may be, in various embodiments, any visible wavelength in the 380-750 nm range. Generally, any suitable type of light source for the given implementation may be selected and used to project light into the cuvette for the purpose of sampling the biomass.

Light sensor 332 is arranged to receive at least some of the light projected by light source 330. In one embodiment, light sensor 332 may be arranged on an opposite side (relative to light source 330) of mixing chamber 314. In some embodiments, light sensor 332 is selected to be particularly sensitive to the wavelengths projected by light source 330, while rejecting other wavelengths.

In the embodiment of FIG. 3, an amount of biomass 334 is present in mixing chamber 314. The illustrated embodiment further shows light source 330 projecting light, as indicated by the arrows extending therefrom into, if not through, mixing chamber 314. Some of the light may pass largely unobstructed through mixing chamber 314 to light sensor 332. Other portions of the light are obstructed (e.g., absorbed) by biomass 334. In practice, it is possible that some amount of light may pass through portions of biomass 334 (e.g., through gaps therein) to light sensor 332, although it is further possible that the intensity of such light may be attenuated. In various embodiments, light sensor 332 may determine an average of the intensity of detected light, with a corresponding indication generated based on the average. In other embodiments, light sensor 332 may be arranged to detect light in both terms of intensity and distribution across the area thereof. With respect to the intensity, light sensor 332 of such an embodiment determines the intensity at which the light was detected. With respect to distribution, light sensor 332 in such an embodiment may determine variations in intensity of detected light, down to a total absence thereof when light is totally absorbed/blocked by the biomass in mixing chamber 314. Generally speaking, light sensor 332 may be any suitable sensor for detecting light in the given application of sensor 130.

In certain embodiments, based on the detected light, light sensor 332 generates an indication, which includes one or more signals that are indicative of both the intensity and distribution of the light. In various embodiments, there is an inverse relationship between the amount of light detected by light sensor 332 (in terms of both intensity and distribution) and the amount of biomass 334 in mixing chamber 314.

Operations of sensor 130 in the embodiment shown may be conducted by controller 340. In the embodiment shown, controller 340 is coupled to relays 310, 318, and 322 associated with valves 306, 316, and 320, respectively. Controller 340 is also coupled to light source 330 and sensor 332. To cause a sample of the biomass to be conducted, controller 340 may cause relay 310 to open valve 306, while keeping valves 316 and 320 shut. Valve 306 may be held open for a specified amount of time and/or until cuvette 302 has received a specified amount of fluid (containing biomass) from bioreactor 100. Upon cuvette 302 having received the specified amount of fluid, controller 340 may change the state of signals conveyed to relay 310 to cause valve 306 to be shut.

After allowing some settling time following the transfer of fluid from bioreactor 100 into cuvette 302, controller 340 continues the sampling process by conveying a signal to light source 330. Responsive to this signal, light source 330 begins projecting light into cuvette 302. If biomass is present in the portion of cuvette 302 in which the light is projected (e.g., mixing chamber 314 in this example), some of this light may be absorbed. As noted above, light source 330 is configured in various embodiments to project light at wavelengths that are absorbed by the expected type of biomass. The light that traverses the entire distance through cuvette 302 may be detected by light sensor 332. The light detected by light sensor 332 results in the generation of an indication that is usable to determine the amount of biomass 334 currently in cuvette 302. The indication in one embodiment comprises signals indicative of the intensity and distribution of light received by light sensor 332.

In certain embodiments, as described above, sensor 130 includes computer system 350 coupled to controller 340. Controller 340 in the embodiment shown forwards the indication received from light sensor 332 to computer system 350. Embodiments are possible and contemplated in which controller 340 performs some pre-processing on the indication prior to forwarding to computer system 350. Upon receiving the indication (pre-processed or not), computer system 350 determines the amount of biomass present in cuvette 302 for the given sample. Computer system 350 may also extrapolate from the amount of biomass determined to be in cuvette 302 to calculate an amount of biomass present in bioreactor 100. Over a number of different samples, computer system 350 may determine a rate of change (e.g., rate of growth) of the amount of biomass in bioreactor 100. For example, if bioreactor 100 is used to generate algae, computer system 350 may determine the rate of growth of the algae, and this information can be used to determine a time to harvest.

In the illustrated embodiment, computer system 350 is coupled to secure processor 220. Secure processor 220 may receive data (e.g., the amount of biomass present in bioreactor 100) from computer system 350. In some embodiments, computer system 350 may be part of secure processor 220. Implementing secure processor 220 may provide secure operations with regards to data measured by sensor 130, as described herein.

Upon completing of a sample in the illustrated embodiment, controller 340 conveys a signal to relay 322 in order to cause the opening of valve 320. When valve 320 is opened, cuvette 302 is drained of fluids contained therein. In this example, the fluid, including the biomass is transferred back to bioreactor 100. Although not explicitly shown, a pump may be implemented between valve 320 and bioreactor 100 to facilitate the transfer of fluid.

A cleaning operation may be performed by sensor 130 subsequent to conducting a sample. After the fluid containing the biomass has been transferred back into bioreactor 100, controller 340 may initiate the cleaning operation by causing relay 318 to open valve 316. When open, valve 316 allows cleaning fluid to be transferred into conduit 312 of cuvette 302. In some embodiments, cleaning fluid source 304 may include a pump or other mechanism to cause the cleaning fluid to be provided at a pressure greater than ambient pressure. The cleaning fluid may be any suitable cleaning fluid, and may be as simple as water. The cleaning fluid may exit cuvette 302 through the bottom of mixing chamber 314 and through valve 320, which remains open during the cleaning operation. In some embodiments, the cleaning fluid (e.g., water) may be transferred to bioreactor 100 and can thus be used as a base for the growth of biomass therein. In other embodiments, additional valves not explicitly shown here may be present to drain the cleaning fluid away from the bioreactor. The cleaning operation may be terminated by controller 340 causing relay 318 to close valve 316 and, subsequently (e.g., after cuvette 302 has been fully drained), causing relay 322 to close valve 320.

In various embodiments, the controller functions carried out by controller 340 may be performed using artificial intelligence (AI) based analysis. For example, machine learning can be used to account for cloudiness in performing samples, where the cloudiness results from biomass adhering to the inner surfaces of cuvette 302 even after cleaning. Various forms of AI-based analysis may also be used to, e.g., adjust the sampling periodicity or other functions related to the automation of the sampling process. The AI/machine learning functions may be carried out on computer system 350 in one embodiment. In another possible embodiment, controller 340 may include functionality capable of carrying out the various AI/machine learning functions. These functions may also be divided between controller 340 and computer system 350 in some embodiments.

In the illustrated embodiments of FIG. 2 and FIG. 3, secure processor 220, generally speaking, is a secure cryptoprocessor, such as a trusted platform module (TPM) or similar hardware device. Typically, secure processor 220 includes physical security measures that render it relatively tamper resistant, and implements cryptographic operations such as the generation and evaluation of cryptographic keys. Collectively, the cryptographic and physical security properties of secure processor 220 facilitate a highly (although not absolutely) trustworthy environment through which measurements generated by sensor 130 are accessed.

Among other functions, secure processor 220 may be configured to cryptographically sign records of measurements made by sensor 130 or other operational records of bioreactor 100. As used herein, cryptographic signing (or simply “signing”) is used in its ordinary sense within the field of cryptography to generally refer to a process of creating a digital signature for a record that enables a recipient to authenticate the signed record (i.e., to verify that the record was created by a known sender) and further enables the recipient to verify that the record was not altered subsequent to signing. Examples of cryptographic signing include public-key or asymmetric cryptography, although any suitable technique may be employed. Among other aspects, cryptographic signing may be broadly understood to involve generating a secure cryptographic hash of a record that is probabilistically highly likely to reveal whether the record has been altered subsequent to signing.

In certain embodiments, sensors 130 include security measures implemented to inhibit tampering or spoofing of data acquired by the sensor. In one embodiment, turning back to FIG. 3, light source 330 in sensor 130 may project light with a particular pattern or other particular component that is received by light sensor 332. For example, light source 330 may flash light in an on/off sequence with a particular timing for the sequence. As another example, light source 330 may change color of the light for a particular amount of time. Inclusion of the particular light pattern or component provided by light sensor 332 may be determined by controller 340. In some embodiments, secure processor 220 provides information regarding the particular light pattern or component to controller 340 for implementation in light source 330.

The particular light pattern or component provided by light source 330 and received by light sensor 332 may be known by secure processor 220 or a server receiving data from the secure processor. The presence of the particular light pattern or component being received in data from light sensor 332 may provide a verification that the data is data collected by sensor 130 (e.g., verify that the data was created by a known sender—sensor 130). In some embodiments, the particular light pattern or component provided by light source 330 may be varied over time to inhibit counterfeiting of the particular light pattern or component. For example, secure processor 220 may randomly or pseudo-randomly generate a different particular light pattern or component provided by light source 330. The randomly or pseudo-randomly generated particular light pattern or component may have a timestamp that secure processor 220 (or a coupled server) recognizes based on the timestamp of data acquired by sensor 130.

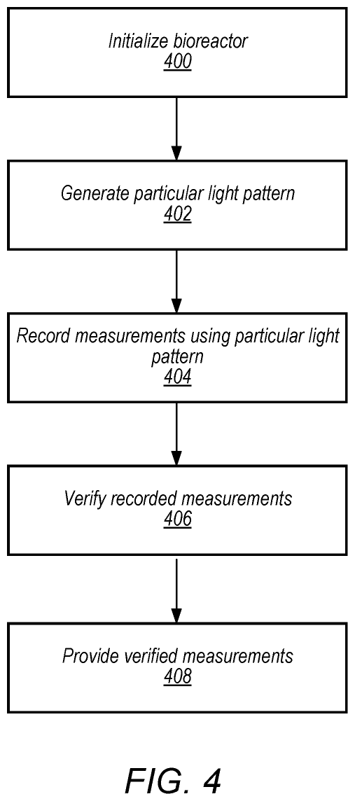

FIG. 4 depicts a flow diagram of an embodiment of a method of operation of bioreactor 100 using sensor 130. Operation begins in block 400 with initialization of bioreactor 100. Initialization, in various embodiments, may include any necessary preparatory or configuration steps needed to prepare the environment of reactor vessel 200 and the control features of bioreactor 100 (e.g., insertion and inoculation of growth medium 210, establishment of defined temperature levels, etc.).

In block 402, bioreactor 100 generates a particular light pattern for use in sensor 130. The particular light pattern may be generated, for example, by secure processor 220 or controller 340. The particular light pattern may be generated in preparation for measurements to be taken by sensor 130. In certain embodiments, the particular light pattern is randomly or pseudo-randomly generated. The particular light pattern may be timestamped when the light pattern is generated.

In block 404, sensor 130 records one or more measurements taken with the particular light pattern. The measurements may be taken using light sensor 332 and processed using computer system 350. In certain embodiments, the measurements are timestamped. For example, secure processor 220 may timestamp individual measurements as they are received. In some embodiments, the received measurements are stored in storage medium 230. In some embodiments, a geographical position of bioreactor 100 are provided for the recorded measurements.

In block 406, the recorded measurements are verified. In certain embodiments, the verification is determined by secure processor 220. Verifying the measurements may include comparing the light patterns in the recorded measurements to the particular light pattern. Comparison of the light patterns in the recorded measurements to the particular light pattern may provide verification on whether the measurements are authentic measurements made by sensor 130. For example, the measurements may be verified by the light patterns in the measurements matching the particular light pattern known by secure processor 220. In some embodiments, timestamps of the recorded measurements are also used in verifying the measurements. For example, the timestamps of the recorded measurements (with light patterns matching the particular light pattern) may be compared to ensure that the measurements were made within a particular time period (e.g., within an acceptable window) from the generation of the particular time pattern (the generation being known by its timestamp). In some embodiments, a geographical position of bioreactor 100 is verified for the recorded measurements to ensure the bioreactor is at its proper location.

In block 408, verified measurements are provided to or released to a controlling entity. The controlling entity may be, for example, an owner of bioreactor 100 or another entity responsible for controlling work related to the bioreactor. The verified measurements may be assessed as “proof of work” for the operator of bioreactor 100 (e.g., proof of physical work by bioreactor 100).

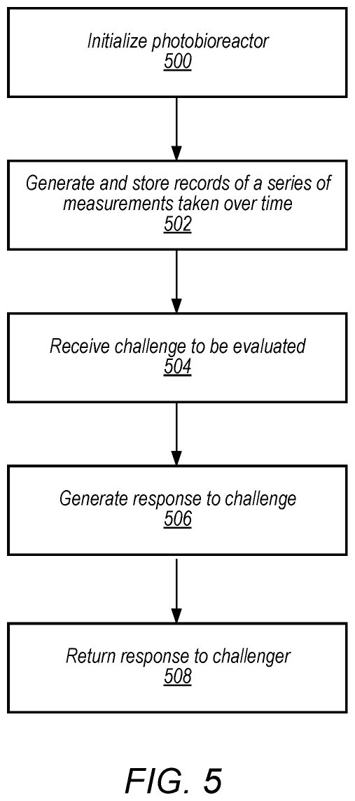

FIG. 5 depicts a flow diagram of an embodiment of a method of operation of bioreactor 100. Operation begins in block 500 with initialization of bioreactor 100. Initialization, in various embodiments, may include any necessary preparatory or configuration steps needed to prepare the environment of reactor vessel 200 and the control features of bioreactor 100 (e.g., insertion and inoculation of growth medium 210, establishment of defined temperature levels, etc.).

In block 502, bioreactor 100 generates and stores records of a series of measurements taken over time. For example, measurements may be performed by sensor 130 and stored within storage medium 230 at intervals that may be regular or randomly/pseudo-randomly determined. In some embodiments, secure processor 220 may sign individual records as they are created. In other embodiments, records may be signed less frequently. For example, some or all records may not be signed until a challenge (discussed below) is received. In some embodiments, geographical position of bioreactor 100 is provided with the taken measurements.

Generally speaking, as elapsed time between creation and signature of a record increases, trust in the reliability of that record decreases, because the record is considerably more vulnerable to having been undetectably modified before it is signed. Nevertheless, operational circumstances may make it unreasonable to expect that every possible record will be signed as soon as it is created. Factors such as the number and/or nature of unsigned records may be taken into account, e.g., by an algorithmic trust model, in determining to what degree the reports from a given bioreactor 100 may be deemed trustworthy.

In some embodiments, bioreactor 100 may also store records of operational events that are not necessarily detected directly by sensor 130. Such events may be detected through the operation of control routines performed within secure processor 220 or elsewhere within bioreactor 100, and may include, e.g., injection of algae, injection of nutrients, changes in lighting, activation of temperature controls (e.g., for heating or cooling), and/or pump activation or deactivation, among other possible events. Records of operational events may or may not be signed by secure processor 220 in various embodiments.

In block 504, bioreactor 100 receives a challenge to be evaluated. As used herein, a “challenge” refers to some type of problem to be solved by bioreactor 100 based at least in part on data that is local to bioreactor 100, such as records of measurements or operational events. A challenge may be implemented with any suitable challenge-response protocol, including public key infrastructure (PKI) techniques, hashes, or the like. In some embodiments, the challenge is initiated by a server or other remote computing system associated with bioreactor 100 or a system of bioreactors.

In block 506, bioreactor 100 generates a response to the challenge. In some embodiments, secure processor 220 will attempt to solve the problem posed by the challenge using the inputs and constraints defined by the challenge. As a simple example, a challenge may require bioreactor 100 to complete the challenge using a current measurement of temperature obtained by sensor 130. The challenge in this example may include a public key, a timestamp, an identifier of the challenge task, and a time constraint (e.g., specifying that the response must be completed within a certain time period from the time represented by the timestamp in order to be considered valid). Secure processor 220 may then, for example, obtain a current temperature measurement from sensor 130 and use the received timestamp as a salt to hash the temperature value. Secure processor 220 may then place the resultant hashed value in a response message, sign the response, and return it to the sender of the challenge. In addition to results generated by evaluating the challenge, the response may also include other data, such as a sensor measurement in unhashed form.

Generally speaking, as with the example just given, the challenge response is a function of both inputs that are supplied to bioreactor 100 in the challenge and data that is local to bioreactor 100. The local data may be dynamically generated in the form of a current measurement that must be completed under a specified time constraint to be valid. The local data may additionally or alternatively employ historically collected data records stored, e.g., within storage medium 230. For example, a challenge may involve generating a response based on a history of measurements made by bioreactor 100.

In some embodiments, the challenge also includes information regarding the state of the sender of the challenge (the “challenger”). Such information may be evaluated by secure processor 220 as part of generating a response to the challenge, and/or may be included in the response to the challenge that is returned to the challenger. For example, secure processor 220 may determine whether the challenge has been validly signed by the challenger prior to processing it.

In block 508, the response is returned to the challenger. The challenger (e.g., server) may in turn evaluate the response to determine whether it is consistent with any input requirements (e.g., timeliness constraints) and whether it solves the challenge in an expected way. Based on these factors, among other possible factors, the challenger may determine a degree of trust to assign to bioreactor 100's response.

Challenges may be received from various possible sources. In some embodiments, bioreactor 100 is capable of sending challenges to itself, e.g., as a form of self-checking. Multiple bioreactors 100 may also be deployed in a system that permits bioreactors 100 to communicate one another. In this instance, one bioreactor 100 may receive a challenge from a different bioreactor 100. Additionally, entities other than bioreactors 100 may submit challenges. For example, remote servers or third-party systems may be employed to audit or otherwise interact with bioreactors 100.

Although the foregoing has been described specifically with respect to sensors deployed in a bioreactor context, this disclosure is not limited to that context. Rather, it is contemplated that the technique of cryptographically signing records of physical measurements may be applied with respect to any type of sensor capable of physical measurements in any suitable context.

The combination of secure processor 220 and sensor 130, along with the challenge-response model described above, generally operates to enhance the level of trust that third parties can place in self-reported physical measurements. As such, the techniques described here yield, for at least some embodiments, a system that enables a device to self-report its proof of physical work in a manner that reduces the likelihood of fabrication, misrepresentation or error. By increasing the reliability of self-reported proof of physical work, these techniques reduce the risk in compensating a user based on such proof. For example, in the case of carbon sequestration via algae production, if bioreactor 100 can be trusted to a reasonable degree that its representations of algae productivity are accurate, then it becomes feasible to issue compensation for that effort. Accordingly, the techniques discussed herein may generally facilitate the monetization of processes that self-report regarding physical work.

Through the techniques discussed above, e.g., the evaluation of a number of challenges and responses, an individual actor (e.g., bioreactor 100) may accumulate a record of “proof” over time that has a corresponding degree of trustworthiness or reliability. For example, some responses may be anomalous, tending to reduce reliability, while others may comport with expectations, tending to increase reliability. Rather than being a binary value, trustworthiness/reliability may be expressed along a range, enabling different levels of discretion for different purposes. For example, if reliability is expressed on a range from 0 to 1, compensation for physical work performed may be scaled according to reliability.

It is noted that in some embodiments, the record of proof developed through self-reported proof of physical work discussed above may be overridden in certain circumstances. For example, some physical processes may have a well-defined lifecycle having a terminal point at which some user intervention is performed to terminate operation and (possibly) initiate a new cycle of operation. In the case of bioreactor 100, algae growth may proceed to a point where the growth needs to be harvested and a new growth cycle initiated. At this point, actual observations regarding the work output may be performed, rather than inferential observations made using sensors 130. For example, the quality of the algae growth may be directly assessed. In such cases, observations regarding final work output may be considered a “final proof of physical work” that overrides the earlier proofs (i.e., is treated as more authoritative). Thus, for example, an actor that seemed unreliable according to its self-reported proof of physical work may ultimately end up producing acceptable work (perhaps because sensor faults led to unreliable measurements) and benefits from the final assessment. Conversely, an actor that provided seemingly reliably reports of proof of physical work but ultimately failed to produce acceptable work would have its reliability downgraded correspondingly.

The foregoing discussion of proof of physical work bears some resemblance to “proof of work” concepts that are applied in other contexts, such as blockchain, spam prevention, and the like. While there are resemblances, there are also important distinctions. Conventional “proof of work” schemes function as barriers to discourage undesirable behavior. They typically consist of computational problems that are easy to verify but moderately burdensome to solve, thus assessing a cost to certain activities. For example, in the context of spam prevention, demanding “proof of work” involves requiring a sender to solve a nontrivial problem. This solution imposes a small burden on legitimate senders who send relatively few messages, but a considerable burden on those who seek to abusively send large volumes of email.

Conventional “proof of work” approaches are essentially entropic. They require an expenditure of effort, and thus energy, to solve a problem for no particular purpose other than to impose a cost on certain kinds of behavior, with the aim of influencing that behavior. By contrast, the “proof of physical work” discussed herein relates to the assessment of measurable change in the physical world. This approach can be applied to evaluate productive effort towards a physical goal that has utility in itself, such as the degree of carbon sequestration performed through algae production. Accordingly, techniques for proof of physical work can be applied to problems that actually reduce entropy (at least locally), as opposed to the entropic nature of conventional “proof of work” approaches. Thus, while there may be some degree of conceptual overlap between proof of physical work as discussed here and conventional “proof of work” approaches, these approaches are fundamentally divergent in important ways.

It is to be understood embodiments disclosed herein are not limited to particular systems described which may, of course, vary. It is also to be understood that the terminology used herein is for the purpose of describing particular embodiments only, and is not intended to be limiting. As used in this specification, the singular forms “a”, “an” and “the” include plural referents unless the content clearly indicates otherwise. Thus, for example, reference to “a sensor” includes a combination of two or more sensors and reference to “a material” includes mixtures of materials.

Further modifications and alternative embodiments of various aspects of the embodiments described in this disclosure will be apparent to those skilled in the art in view of this description. Accordingly, this description is to be construed as illustrative only and is for the purpose of teaching those skilled in the art the general manner of carrying out the embodiments. It is to be understood that the forms of the embodiments shown and described herein are to be taken as the presently preferred embodiments. Elements and materials may be substituted for those illustrated and described herein, parts and processes may be reversed, and certain features of the embodiments may be utilized independently, all as would be apparent to one skilled in the art after having the benefit of this description. Changes may be made in the elements described herein without departing from the spirit and scope of the following claims.

What is claimed is:

1. An apparatus, comprising:
a bioreactor vessel;
at least one sensor configured to perform a physical measurement of the bioreactor vessel; and
a secure processor coupled to the at least one sensor, the secure processor being configured to cryptographically sign a record of the physical measurement.

2. The apparatus of claim 1, further comprising a storage medium configured to store records of a plurality of physical measurements.

3. The apparatus of claim 2, wherein the secure processor is configured to cryptographically sign individual records of the plurality of physical measurements when corresponding individual ones of the plurality of physical measurements are performed.

4. The apparatus of claim 2, wherein the secure processor is configured to sign multiple records of the plurality of physical measurements at a time after physical measurements corresponding to the multiple records have been performed.

5. The apparatus of claim 1, wherein the secure processor is configured to receive a challenge and generate a response to the challenge based at least in part upon the record of the physical measurement.

6. The apparatus of claim 5, wherein, to generate the response to the challenge, the secure processor is configured to perform an operation specified by the challenge using the record of the physical measurement as an input.

7. The apparatus of claim 5, wherein, to generate the response to the challenge, the secure processor is configured to evaluate information identifying a sender of the challenge.

8. The apparatus of claim 7, wherein the information identifying the sender of the challenge is included within the challenge.

9. The apparatus of claim 5, wherein, to generate the response to the challenge, the secure processor is configured to determine whether the challenge has been validly signed by a sender of the challenge.

10. The apparatus of claim 5, wherein, to generate the response to the challenge, the secure processor is configured to cryptographically sign the response to the challenge.

11. The apparatus of claim 10, wherein to cryptographically sign the response to the challenge, the secure processor is configured to generate a signature based at least on a public key included within the challenge.

12. The apparatus of claim 1, wherein the bioreactor vessel stores a growth medium.

13. The apparatus of claim 12, wherein the growth medium is selected to support growth of one or more selected species of algae.

14. The apparatus of claim 12, wherein the at least one sensor is configured to perform physical measurements of characteristics of the growth medium.

15. An apparatus, comprising:
a bioreactor vessel containing a growth medium;
a sensor configured to perform a physical measurement of the growth medium in the bioreactor vessel, wherein the sensor includes a light source and a light sensor; and
a secure processor coupled to the sensor, the secure processor being configured to generate a particular light pattern for projection using the light source.

16. The apparatus of claim 15, wherein the secure processor is configured to verify the physical measurement based on assessment that the particular light pattern is in light received by the light sensor.

17. The apparatus of claim 15, wherein the particular light pattern includes an on/off sequence for the light source with a particular timing for the on/off sequence.

18. The apparatus of claim 15, wherein the particular light pattern includes a change in color of light from the light source for a particular amount of time.

19. The apparatus of claim 15, wherein the secure processor is configured randomly or pseudo-randomly generate the particular light pattern.

20. The apparatus of claim 15, wherein the sensor is configured to provide a timestamp for the physical measurement, and wherein the secure processor is configured to verify that the timestamp for the physical measurement is within an acceptable window.
